(19) United States
(12) Patent Application Publication
(10) Pub. No.: US 2003/0017137 A1
(43) Pub. Date: Jan. 23, 2003

(54) VPR-DRIVEN DNA OR RNA CONSTRUCT AND THERAPEUTIC USES THEREOF

(76) Inventors: CAROLINE ALFIERI, DORION (CA); JEROME TANNER, DORION (CA); PHILIPPE ROUX, MONTREAL (CA)

Correspondence Address: SWABEY OGILVY RENAULT, SUITE 1600, 1981 MCGILL COLLEGE AVENUE, MONTREAL, H3A2Y3, CA

(21) Appl. No.: 09/120,286

(22) Filed: Jul. 22, 1998

(51) Int. Cl. A61K 48/00; C07H 21/04; A61K 39/21; C12N 5/06

(52) U.S. Cl. 424/93.1; 424/188.1; 435/320.1; 435/328; 536/23.53; 536/24.1

(57) ABSTRACT

The present invention relates to a DNA or RNA construct capable of expression of IL-2 in a warm-blooded animal or biological preparation, the recombinant DNA or RNA construct comprising a) a Vpr activated promoter; b) a transcribable DNA segment coding for IL-2 and; c) a secretory DNA encoding for a signal peptide functional in mammary cells and operably linked between the promoter and the DNA segment to facilitate secretion of the IL-2. The present invention also relates to a method for increasing the immune response of a warm-blooded animal or biological preparation. There is also described methods for inhibiting or stimulating expression of IL-8 of a warm-blooded animal or a biological preparation.

### BACKGROUND OF THE INVENTION

(a) Field of the Invention

The present invention relates to Vpr-driven DNA or RNA construct and therapeutic uses thereof. The present invention also relates to methods for stimulating the immunity of warm-blooded animals and more particularly of immunocompromised patients.

(b) Description of the Prior Art

Macrophages and monocytes are major targets of the human immunodeficiency virus type 1 (HIV-1), serving both as reservoirs for long-term virus production and as vehicles for viral dissemination throughout the body. Replication of HIV in tissue macrophages has been associated with clinical manifestations of disease, including encephalopathy, vacuolar myelopathy, pneumonitis and lymphatic hyperplasia.

In non-HIV settings, monocytes/macrophages act as early sentries to bacterial and viral infections, serving to alert and link the various arms of the immune system through the expression of proinflammatory cytokines. HIV has unfortunately found several ways to subvert the expression of many of these same proinflammatory cytokines, resulting in their dysregulated secretion. This dysregulated cytokine expression can in turn contribute to increased HIV replication and additional pathogenesis associated with the HIV disease state.

Interleukin-8 (IL-8) is one of several proinflammatory cytokines expressed during the inflammatory response to infection. IL-8 was first identified as a 72 amino acid neutrophil chemotactic polypeptide. Subsequent studies have found that IL-8 exhibits chemotactic activities against basophils and T lymphocytes, induces neutrophils to release lysosomal enzymes, to increase their expression of Mac-1 and CR-1, and to adhere to endothelial cells by the increased expression of VCAM-1 and L-selectins. Although IL-8 is produced by a variety of cell types, monocytes and macrophages constitute the most prominent source of this cytokine.

IL-8 serum levels are significantly increased in HIV-infected individuals (34-fold). In vitro IL-8, along with IL-6 and TNFα, are induced early post-HIV-monocytic-infection, followed by their continued increased expression. Interestingly, studies on the in vitro expression of IL-8 in HIV-infected monocytes found a close correlation between extracellular IL-8 levels and the levels of HIV p24. This might suggest that a lytic-cycle HIV protein or proteins may be involved in the observed increase in IL-8 expression.

The HIV protein Vpr is a highly conserved viral auxiliary protein encoded by the open reading frame R found in both human and simian immunodeficiency viruses. The vpr gene encodes a 14-kDa, 96 amino acid protein which is expressed primarily from a singly spliced Rev-dependent mRNA, and able to form post-translational oligomeric species in vitro. Vpr is typically assembled in the virus particle and, upon virus entry and uncoating, participates with the viral matrix proteins in targeting the HIV pre-integration complex to the cell nucleus. When expressed alone, Vpr has been shown to cause cells to accumulate in the G2/M phase of the cell cycle by blocking the activation of the mitogenic cyclin-dependent kinase. Vpr has also been shown to induce cellular differentiation, promote cell apoptosis and transactivate several heterologous viral promoters (Cohen, E. A. et al., 1990, J. AIDS 3:11-18). Vpr has been found associated with several cellular proteins, one of which has been tentatively identified as belonging to the glucocorticoid receptor (GR) family.

Given the ability of HIV to increase the synthesis of proinflammatory cytokines in monocytes and macrophages concomitant with the time of increased virus replication, the ability of Vpr to induce cell differentiation and to associate with cellular signal transduction molecules (i.e. GR) which in turn have the potential to alter cytokine expression, we undertook experiments to determine whether Vpr could alter the expression of a proinflammatory molecule, in particular IL-8. We chose IL-8 as our prototypic proinflammatory cytokine based on its increased expression following HIV infection, its potential regulation by GR (IL-8 contains a putative GR-responsive element within its promoter sequence), as well as the presence of enhancer elements in the IL-8 promoter common to several other HIV-dysregulated proinflammatory cytokines (Mukaida, N. et al., 1989, J. Immunol. 143:1366-1371). Results indicate that Vpr is capable of inducing IL-8 protein expression, acting chiefly through the stimulation of NF-IL-6/NF-κB transcription factors.

It would be highly desirable to be provided with methods to effectively stimulate the immune response in immunocompromised patients affected by HIV, cancer and other immunocompromising diseases.

### SUMMARY OF THE INVENTION

One aim of the present invention is to provide methods for stimulating the immune response of a warm-blooded animal or biological preparation.

Another aim of the present invention is to provide methods for increasing the immune response of immunocompromised patients.

Another aim of the present invention is to provide DNA or RNA constructs useful to effectively increase the immune response of a warm-blooded animal or biological preparation.

Another aim of the present invention is to provide DNA or RNA constructs useful to effectively increase the immune response of an immunocompromised patient.

Another aim of the present invention is to provide methods for determining the interaction between Vpr and other proteins.

Another aim of the present invention is to provide methods for inhibiting IL-8 expression.

Another aim of the present invention is to provide methods for stimulating IL-8 expression.

In accordance with the present invention there is provided a DNA or RNA construct capable of expression of IL-2 in a warm-blooded animal or biological preparation said recombinant DNA or RNA construct comprising:

a) a Vpr activated promoter;

b) a transcribable DNA segment coding for IL-2; and

c) a secretory DNA encoding for a signal peptide functional in mammary cells and operably linked between said promoter and said DNA segment to facilitate secretion of said IL-2.

In a preferred embodiment the DNA or RNA construct is recombinant.

In a preferred embodiment the promoter is NFκB, NF-IL-6, or NFκB/NF-IL-6.

In an alternative embodiment, the promoter sequence comprises the bases corresponding to -94 to -72 of the NFκB/NF-IL-6 enhancer sequence.

In an alternative embodiment the recognition sequences of the promoter is selected from the group consisting of:

NFκB recognition sequence:

AGGAAATTCCA (SEQ ID NO:1);

NF-IL-6 recognition sequence:

CAGTTGCAAATCGTG (SEQ ID NO:2);

NF-IL-6/NFκB recognition sequence:

AGTTGCAAATCGTGGAATTTCCTGAA (SEQ ID NO:3) and;

-94 to -72 of the IL-8 core enhancer element NF-IL-6.NFκB recognition sequence:

CAGTTGCAAATCGTGGAATTTCC (SEQ ID NO:4).

In a preferred embodiment said transcribable DNA is the IL-2 cDNA sequence (Devos, R. et al., 1983, Nucl. Ac. Res. 11:4307-4323).

In a more preferred embodiment the transcribable IL-2 cDNA has the following sequence:

| | | |
|---|---|---|
| CCCCATAATA TTTTTCCAGA ATTAACAGTA TAAATTGCAT CTCTTGTTCA AGAGTTCCCT | 60 | (SEQ ID NO:5) |
| ATCACTCTCT TTAATCACTA CTCACAGTAA CCTCAACTCC TGCCACAATG TACAGGATGC | 120 | |
| AACTCCTGTC TTGCATTGCA CTAAGTCTTG CACTTGTCAC AAACAGTGCA CCTACTTCAA | 180 | |
| GTTCTACAAA GAAAACACAG CTACAACTGG AGCATTTACT GCTGGATTTA CAGATGATTT | 240 | |
| TGAATGGAAT TAATAATTAC AAGAATCCCA AACTCACCAG GATGCTCACA TTTAAGTTTT | 300 | |
| ACATGCCCAA GAAGGCCACA GAACTGAAAC ATCTTCAGTG TCTAGAAGAA GAACTCAAAC | 360 | |
| CTCTGGAGGA AGTGCTAAAT TTAGCTCAAA GCAAAAACTT TCACTTAAGA CCCAGGGACT | 420 | |
| TAATCAGCAA TATCAACGTA ATAGTTCTGG AACTAAAGGG ATCTGAAACA ACATTCATGT | 480 | |
| GTGAATATGC TGATGAGACA GCAACCATTG TAGAATTTCT GAACAGATGG ATTACCTTTT | 540 | |
| GTCAAAGCAT CATCTCAACA CTGACTTGAT AATTAAGTGC TTCCCACTTA AAACATATCA | 600 | |
| GGCCTTCTAT TTATTTAAAT ATTTAAATTT TATATTTATT GTTGAATGTA TGGTTTGCTA | 660 | |
| CCTATTGTAA CTATTATTCT TAATCTTAAA ACTATAAATA TGGATCTTTT ATGATTCTTT | 720 | |
| TTGTAAGCCC TAGGGGCTCT AAAATGGTTT CACTTATTTA TCCCAAAATA TTTATTATTA | 780 | |
| TGTTGAATGT TAAATATAGT ATCTATGTAG ATTGGTTAGT AAAACTATTT AATAAATTTG | 840 | |
| ATAA | 844 | |

In an alternative embodiment the DNA or RNA construct of the present invention further comprises a trancribable segment of IL-8.

In a more preferred embodiment the transcribable IL-8 cDNA has the following sequence:

| | | |
|---|---|---|
| GAATTCAGTA ACCCAGGCAT TATTTTATCC TCAAGTCTTA GCTTGGTTGG AGAAAGATAA | 60 | (SEQ ID NO:6) |
| CAAAAAGAAA CATGATTGTG CAGAAACAGA CAAACCTTTT TGGAAAGCAT TTGAAAATGG | 120 | |
| CATTCCCCCT CCACAGTGTG TTCACAGTGT GGGCAAATTC ACTGCTCTGT CGTACTTTCT | 180 | |
| GAAAATGAAG AACTGTTACA CCAAGGTGAA TTATTTATAA ATTATGTACT TGCCCAGAAG | 240 | |
| CGAACAGACT TTTACTATCA TAAGAACCCT TCCTTGGTGT GCTCTTTATC TACAGAATCC | 300 | |
| AAGACCTTTC AAGAAAGGTC TTGGATTCTT TTCTTCAGCA CACTAGGACA TAAAGCCACC | 360 | |
| TTTTTATGAT TTGTTGAAAT TTCTCACTCC ATCCCTTTTG CTGATGATCA TGGGTCCTCA | 420 | |
| GAGGTCAGAC TTGGTGTCCT TGGATAAAGA GCATGAAGCA ACAGTGGCTG AACCAGAGTT | 480 | |
| GGAACCCAGA TGCTCTTTCC ACTAAGCATA CAACTTTCCA TTAGATAACA CCTCCCTCCC | 540 | |
| ACCCCAACCA AGCAGCTCCA GTGCACCACT TTCTGGAGCA TAAACATACC TTAACTTTAC | 600 | |
| AACTTGAGTG GCCTTGAATA CTGTTCCTAT CTGGAATGTG CTGTTCTCTT TCATCTTCCT | 660 | |
| CTATTGAAGC CCTCCTATTC CTCAATGCCT TGCTCCAACT GCCTTTGGAA GATTCTGCTC | 720 | |
| TTATGCCTCC ACTGGAATTA ATGTCTTAGT ACCACTTGTC TATTCTGCTA TATAGTCAGT | 780 | |
| CCTTACATTG CTTTCTTCTT CTGATAGACC AAACTCTTTA AGGACAAGTA CCTAGTCTTA | 840 | |
| TCTATTTCTA GATCCCCCAC ATTACTCAGA AAGTTACTCC ATAAATGTTT GTGGAACTGA | 900 | |
| TTTCTATGTG AAGACATGTG CCCCTTCACT CTGTTAACTA GCATTAGAAA AACAAATCTT | 960 | |
| TTGAAAAGTT GTAGTATGCC CCTAAGAGCA GTAACAGTTC CTAGAAACTC TCTAAAATGC | 1020 | |
| TTAGAAAAAG ATTTATTTTA AATTACCTCC CCAATAAAAT GATTGGCTGG CTTATCTTCA | 1080 | |
| CCATCATGAT AGCATCTGTA ATTAACTGAA AAAAAATAAT TATGCCATTA AAAGAAAATC | 1140 | |
| ATCCATGATC TTGTTCTAAC ACCTGCCACT CTAGTACTAT ATCTGTCACA TGGTCTATGA | 1200 | |
| TAAAGTTATC TAGAAATAAA AAAGCATACA ATTGATAATT CACCAAATTG TGGAGCTTCA | 1260 | |
| GTATTTTAAA TGTATATTAA AATTAAATTA TTTTAAAGAT CAAAGAAAAC TTTCGTCATA | 1320 | |
| CTCCGTATTT GATAAGGAAC AAATAGGAAG TGTGATGACT CAGGTTTGCC CTGAGGGGAT | 1380 | |
| GGGCCATCAG TTGCAAATCG TGGAATTTCC TCTGACATAA TGAAAAGATG AGGGTGCATA | 1440 | |
| AGTTCTCTAG TAGGGTGATG ATATAAAAAG CCACCGGAGC ACTCCATAAG GCACAAACTT | 1500 | |
| TCAGAGACAG CAGAGCACAC AAGCTTCTAG GACAAGAGCC AGGAAGAAAC CACCGGAAGG | 1560 | |
| AACCATTCTC ACTGTGTGTA AACATG | 1586 | |

In a preferred embodiment the signal peptide is an IL-2 signal peptide or an analogue or derivative thereof.

In a preferred embodiment the IL-2 signal peptide has the following sequence:

| Met Tyr Arg Met Gin Leu Leu Ser Cys Ile Ala Leu Ser Leu Ala Leu | (SEQ ID NO:7) |
|---|---|
| 1 5 10 15 | |
| Val Thr Asn Ser Ala Pro Thr Ser Ser Ser | |
| 20 25 | |

In a further aspect of the present invention, there is provided a method for increasing the immune response of a warm-blooded animal or biological preparation comprising the steps of:

a) introducing a DNA or RNA construct as defined in accordance with the present invention in stem cells, antigen presenting cells or immune cell leukocytes, fibroblasts and epithelial cells, of the warm-blooded animal or biological preparation to obtain transfected cell populations mentioned above; and

b) administering a pharmaceutically effective amount of said transfected cell populations to the warm-blooded animal or biological preparation.

Within another aspect of the present invention, methods are provided for delivering DNA or RNA constructs to a warm-blooded animal or biological preparation, wherein the DNA or RNA construct directs the expression of IL-2.

In another aspect of the present invention, there is provided a method for inhibiting expression of IL-8 of a warm-blooded animal or biological preparation comprising the step of administering a pharmaceutically effective amount of a Vpr inhibitor.

In a preferred embodiment, the Vpr inhibitor is an anti-Vpr antibody.

The anti-Vpr antibody can be monoclonal or polyclonal.

In another aspect of the present invention, there is provided, a method for stimulating IL-8 expression in a mammal in need to, said method comprising the step of administering a pharmaceutically effective amount of a pharmaceutically acceptable formulation comprising a Vpr protein to said mammal.

Preferably, the warm-blooded animal is an immunocompromised patient.

In another aspect of the present invention, there is provided a method for determining the interaction between Vpr and other proteins, said method comprises the steps of

a) co-precipitation of Vpr and associated cellular proteins using anti-Vpr antibody followed by protein gel electrophoresis;

b) development of a yeast two hybrid system in which a Vpr-Gal4 construct is introduced into yeast to screen human cDNAs expressed in a yeast library and detection of Gal4 insensitive colonies; and

c) construction of Vpr deletion mutants to identify both association of cellular proteins with Vpr or Vpr subdomains.

### BRIEF DESCRIPTION OF THE DRAWINGS

FIG. 1 illustrates the optimization of the transient expression system in A549 cells;

FIG. 2 illustrates the expression of HIV-1 Vpr in transfected COS-7 and A549 cells;

FIG. 3 illustrates the effect of HIV-1 Vpr expression on the secretion of IL-8;

FIG. 4 illustrates the effect of Vpr on the activity of the IL-8 promoter in IL-8 inducible cell-lines; and

FIG. 5 illustrates the identification of Vpr-responsive elements required for the transcriptional activation of the IL-8 gene.

### DETAILED DESCRIPTION OF THE INVENTION

Prior to setting forth this invention it may be helpful to first define certain terms that will be used herein.

“Biological preparation” refers to any animal cell or tissue ex vivo. Suitable preparations include, by way of example, HepG2 cells, COS cells, 293 cells, and ATT20 cells.

The term “DNA or RNA constructs” refers to a naturally occurring DNA or RNA which has been modified or to an assembly which directs the expression of a gene of interest. The DNA or RNA construct must include promoter elements, and a sequence which, when transcribed, is operably linked to the gene of interest and acts as a translation initiation sequence. The DNA or RNA construct may also include a signal which directs poly-adenylation, one or more selectable markers, as well as one or more restriction sites.

Vpr means Human Immunodeficiency Virus Type 1 protein R.

By the term “Vpr activated promoter” is meant a promoter which can be activated by the Vpr protein. Examples of Vpr promoters include but are not limited to NFκB, NF-IL-6, and NFκB/NFIL-6. Preferably the promoter comprises at least the sequence corresponding to IL-8-94 to -72 which encodes the NF-IL-6/NFκB enhancer sequence.

By the terms Vpr protein” or “Vpr” are meant proteins which have the biological activity of the Vpr protein. These terms also include biologically active analogues of the Vpr protein such as recombinant proteins, and proteins in which one or more amino acids have been deleted, replaced or modified by methods well known in the art of protein synthesis.

By the term “stem cells” is meant a progenitor or precursor cell from which more specialized cell types are derived. Stem cells have the capacity to be pluripotent. Antigen presenting cells are highly specialized cells that can process antigens and display their peptide fragments on the cell surface together with molecules required for lymphocyte activation. These can include dendritic cells, macrophages, B cells, and epithelial cells.

By the term “Vpr inhibitor” is meant a compound which interacts with the Vpr protein to inhibit its biological activity. Examples of Vpr inhibitors include Vpr-specific binding proteins or peptides derived from said proteins, pharmacological and drug agents which interact with Vpr directly to block Vpr binding to cellular proteins, or block Vpr activation of Vpr responsive promoters.

Vpr inhibitors include monoclonal and polyclonal anti-Vpr antibodies.

The DNA or RNA constructs and Vpr inhibitors may be administered by injection, infusion, orally, rectally, lingually, or transdermally. Depending on the mode of administration, the compounds or separate components can be formulated with the appropriate diluents and carriers to form ointments, creams, foams, and solutions.

Injection may be intravenous, intramuscular, intracerebral subcutaneous, or intraperitoneal. For injection or infusion, the compound would be in the form of a solution or suspension. It would be dissolved or suspended in a physiologically compatible solution in a therapeutically effective amount.

For oral administration, the compounds may be in capsule, table, oral suspension, or syrup form. The tablet or capsules would contain a suitable amount to comply with the general and preferred ratios set forth below. The capsules would be the usual gelatin capsules and would contain, in addition to the three compounds, a small quantity of magnesium stearate or other excipient.

Tablets would contain a therapeutically effective amount of the compound and a binder, which may be a gelatin solution, a starch paste in water, polyvinyl pyrilidone, polyvinyl alcohol in water or any other suitable binder, with a typical sugar coating.

Syrup would contain a therapeutically effective amount of the compound.

The terms “stimulating the immune response”, “inhibiting IL-8 expression” and “stimulating IL-8 expression” as used within the context of the present invention refer to a stimulation or an inhibition of a response when compared to an untreated control.

IL-2, Vpr, and IL-8 cDNA may be prepared as the gene of interest by obtaining either in full length or truncated mutants cloned from mammalian cDNA using any one of several methods described in Sambrook et al., Molecular Cloning: A Laboratory Handbook, Cold Springs Harbor Press (1989). In the context of the present invention, the gene of interest is composed of a portion of the gene encoding IL-2 or IL-8 which, when expressed, would disrupt the normal functioning of the cells.

A wide variety of methods may be utilized in order to deliver the DNA or RNA constructs of the present invention to a warm-blooded animal or biological preparation. For example, within one embodiment of the invention, the vector construct is inserted into a retroviral vector, which may then be administered directly into a warm-blooded animal or biological preparation. Representative examples or suitable retroviral vectors and methods are described in more detail in the following U.S. patents and patent applications, all of which are incorporated by reference herein in their entirety: “DNA constructs for retrovirus packaging cell lines”, U.S. Pat. No. 4,871,719; “Recombinant Retroviruses with Amphotropic and Ectotropic Host Ranges”, PCT Publication No. WO 90/02806; and “Retroviral Packaging Cell Lines and Processes of Using Same”, PCT Publication No. WO 89/07150.

DNA or RNA constructs may also be carried by a wide variety of other viral vectors, including for example, recombinant vaccinia vectors (U.S. Pat. Nos. 4,603,112 and 4,769,330), recombinant pox virus vectors (PCT Publication No. WO 89/01973), poliovirus (Evans et al. Nature, 339:385-388 (1989); and Sabin, J. Biol. Standardization, 1:115-118 (1973)); influenza virus (Luytjes et al., Cell, 59:1107-1113 (1989); McMichael et al., N. Eng. J. Med., 309:13-17 (1983); and Yap et al., Nature, 273:238-239 (1978)); adenovirus (Berkner, Biotechniques, 6:616-627 (1988); Rosenfeld et al., Science, 252:431-34 (1991)); adeno-associated virus (Samulski et al., J. Virol., 63:3822-3828 (1989); Mendelson et al. , Virol. , 166:154-165 (1988)); herpes (Kit, Avd. Exp. Med. Biol., 215:219-236 (1989)); and HIV (Poznansky, J. Virol., 65:532-536 (1991)).

In addition, DNA or RNA constructs may be administered to warm-blooded animals or biological preparations utilizing a variety of physical methods, such as lipofection (Felgner et al. Proc. Natl. Acad. Sci. USA, 84:7413-7417 (1989), direct DNA injection (Acsadi et al., Nature, 352:815-818 (1991)); microprojectile bombardment (Williams et al., PNAS, 88-2726-2730 (1991)); liposomes (Wang et al., PNAS, 84:7851-7855 (1987)); CaPO4 (Dubensky et al., PNAS, 81:7529-7533 (1984)); or DNA ligand (Wu et al., J. Biol. Chem., 264:16985-16987 (1989)).

A therapeutic amount may be determined by in vitro experimentation followed by in vivo studies.

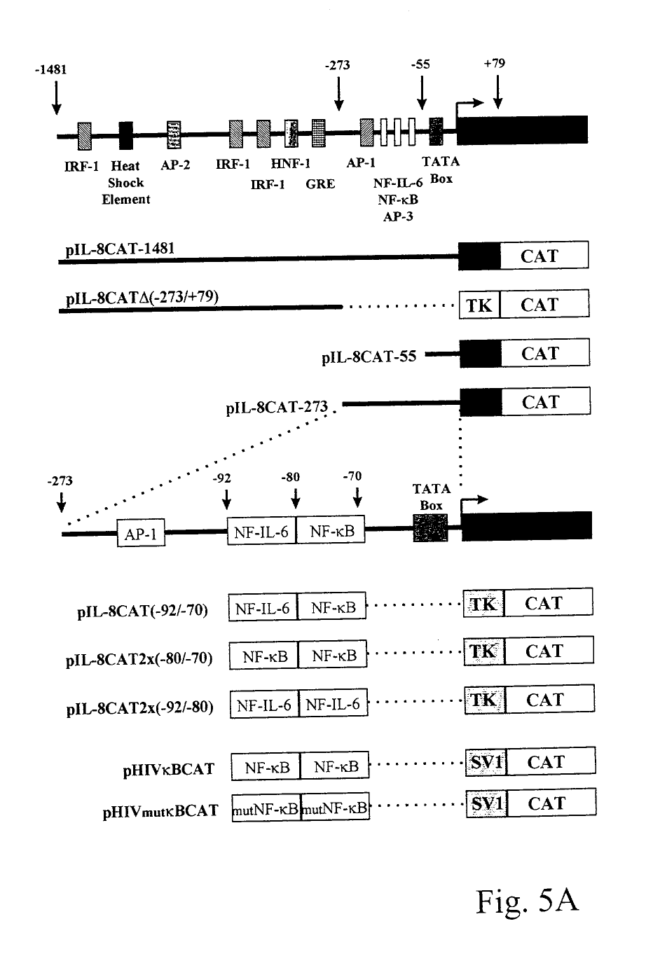

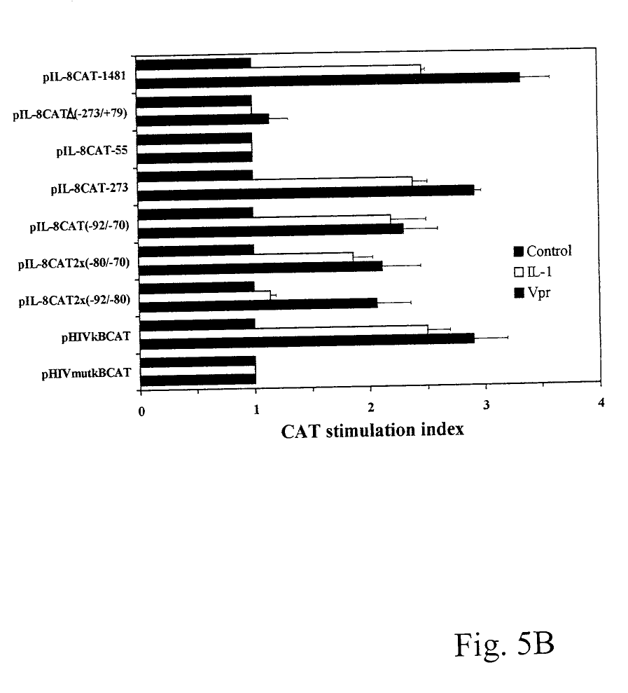

In accordance with the present invention it was found that the HIV protein Vpr is capable of increasing the expression of the proinflammatory cytokine IL-8 through activation of NF-κB and NF-IL-6 transcription factors. Although previous results indicated that Vpr can associate with the glucocorticoid receptor, and a modest transcriptional enhancement of CAT activity was also observed with IL-8 promoter construct -1481 to -273 which contains a glucocorticoid responsive element, the level of CAT transcription in these IL-8 promoters was markedly less (0.3-fold) as compared with full length IL-8 promoter constructs (4-fold) (FIG. 5B) or with constructs containing NF-IL-6/NF-κB enhancer elements (2 to 3 fold) (FIG. 5B). This is the first report on Vpr acting as a stimulator of NF-κB or NF-IL-6 for induction of a proinflammatory cytokine.

In addition to the IL-8 promoter, it was found that several other promoters containing NF-κB (IL-2, HIV-LTR and IL-2R) or KB-like sequences (CMV and SV40) demonstrated enhanced transcriptional activity when co-transfected with a Vpr expression plasmid. The enhanced transcription appeared specific, since use of the TK promoter, which does not contain KB-like sequences, did not show significant increases in CAT activity.

Determination of a common in vitro and in vivo role or function for Vpr has been somewhat controversial. Although dispensable for in vitro HIV replication, Vpr appears essential for virus replication in vivo, and is conserved in both human and simian strains of virus. HIV and SIV obtained following culture passage often encode truncated versions of Vpr, whereas laboratory derived HIV strains containing point mutations were observed to have high rates of reversion to wild type in infected macaques. Additional in vitro evidence using mutant Vpr containing viruses demonstrated slower replication as compared to wild-type virus on T cell lines and primary T cells and particularly poor replication in monocytes. Finally, antisense Vpr oligodeoxynucleotides have been shown to inhibit replication of HIV in primary macrophages and exogenous Vpr was active in promoting HIV replication in T cells and monocytes particularly in newly infected monocytic cells, a finding consistent with mutational studies.

In addition to the differential need for Vpr in vitro and in vivo, Vpr also exhibited G2/M growth inhibition and the ability to induce apoptosis. Given the fact that HIV replicates more readily in mitogenically induced primary cells, these G2/M growth-inhibitory and apoptosis-promoting abilities of Vpr would appear incongruous with the need for the virus to replicate. In fact, both of these attributes would appear to reduce virus titer and the ability of the virus to persist in cells. The results of the present application indicate that virus replication may continue in the presence of Vpr's growth-arresting action through the induction of NF-κB. NF-κB is normally present in the cytoplasm as a heterodimer composed of proteins from the Rel family (c-Rel, Rel A and Rel B; p65), NF-κB 1 and NF-κB2 (p50, p52) complexed with the inhibitor protein IKB (reviewed in 49). Upon release of NF-κB from IKB, the dimer is transported to the nucleus. Interestingly, NF-kB was shown to be regulated by cyclin-dependent kinases and the cell cycle, suggesting that the Vpr-induced cell cycle arrest at the G2/M interphase may promote increased levels of NF-κB.

In addition to IL-8, other genes including angiotensinogen, IL-6 and serum amyloid A1 contain adjacent binding sites for NF-κB and NF-IL-6. In addition, Dunn et al. (1994) have recently described the cooperative binding of RelA and NF-IL-6 in the TNFα responsive region of the G-CSF promoter. Consequently it would appear that a number of genes involved in the proinflammatory response and induced following HIV infection may be controlled by Vpr.

In conclusion, the present inventors surprisingly found that Vpr expression and subsequent activation of NF-κB and/or NF-IL-6 lead to increased expression of the proinflammatory cytokine IL-8 and to HIV-LTR transcription.

Other characteristics and advantages of the present invention will appear from the following examples. The following examples are intended to document the invention, without limiting its scope.

The present invention will be more readily understood by referring to the following examples which are given to illustrate the invention rather than to limit its scope.

### EXAMPLE 1

### Vpr Increases the Expression of Proinflammatory Cytokine Interleukin-8

Material and Methods

Reagents and Antiserum.

Recombinant IL-1a was obtained from Boehringer Mannheim (Laval, PQ). Rabbit anti-Vpr serum (kind gift from Dr. E. Cohen, University of Montreal) was obtained using bacterially derived Vpr recombinant protein (HIV strain ELI), as previously described.

Cell Culture.

The monocytic cell line U937 (CRL-1593, American Type Culture Collection, Rockville, Md.), HL-60 (CCL-240, ATCC) and the T-lymphoid cell line Jurkat (TIB-152, ATCC) were maintained in RPMI 1640 (Gibco BRL, Burlington, ON) supplemented with 10% heat-inactivated fetal calf serum (FCS, Gibco BRL), 100 U/ml penicillin and 80 μg/ml gentamicin. The human A549 pulmonary epithelial cells (kindly provided by Dr. B. Massie, National Research Council of Canada, Montreal, Qc) and the African green monkey kidney cell line, COS-7 (CRL-1651, ATCC) were maintained in Dulbecco modified Eagle medium (Gibco BRL) supplemented with 10% heat-inactivated FCS, 100 U/ml penicillin and 80 μg/ml gentamicin.

Plasmid Construction.

The various IL-8 promoter constructs used to map Vpr-responsive IL-8 promoter elements were constructed with PCR amplification products using synthetic primers within the IL-8 promoter region, namely (-1481 5′ -GAATTCAGTAACCCAGGCAT (SEQ ID NO:8)), (-55 5′-GATGAGGGTGCATAAGTTCTC (SEQ ID NO:9)) and (+79 5′-GAATTCAGTAACCCAGGCAT (SEQ ID NO:8)), unique internal restriction sites within the IL-8 promoter (Xba I site at position -273) or chemically synthesized sites (2x(-80/-70) 5′-gggaagcttTGGAATTTCCTTGGAATTTCCTgggatccgg (SEQ ID NO:10)), (2x(-92/-80)-5′ -gggaagcttCAGTTGCAAATCGTGGCAGTTGCA AATCGTgggatccgg (SEQ ID NO:11)) or ((-92/-70)-5′-gggaagcttCAGTTGCAAATCGTGGAATTTCCTggatccggg (SEQ ID NO:12)), NF-κB, NF-IL-6 or NF-κB/NF-IL-6, respectively, using an Applied Biosystems 394 DNA/RNA nucleotide synthesizer and based on the human IL-8 promoter sequence. The resulting PCR fragments or synthetic DNAs were subcloned into the EcoRI site of PCR cloning vector pCR2.1 using the TA cloning kit (Invitrogen, Carlsbad, Calif.), or the HindIII/BamHI site of pUC19, followed by subsequent insertion into the chloramphenicol acetyltransferase (CAT) reporter plasmids pBLCAT3 or pBLCAT2, respectively. All plasmids constructed by PCR amplification were verified for sequence fidelity by DNA sequencing according to the published sequence (Mukaida, N. et al., 1989, J. Immunol. 143:1366-1371). PSVCMVER is an HIV (strain ELI) Vpr eucaryotic expression plasmid driven by the CMV IE promoter as previously described. Heterologous promoter-CAT plasmid constructs, namely, pHIV/CAT, pHIVkBCAT and pHIVmutkBCAT, PCMVCAT, pSV2CAT and pA10CAT₂, pIL-2CAT, pIL-2RαCAT, and pTKCAT, were used to determine additional Vpr responsive elements, and have been previously described.

Transient Transfection.

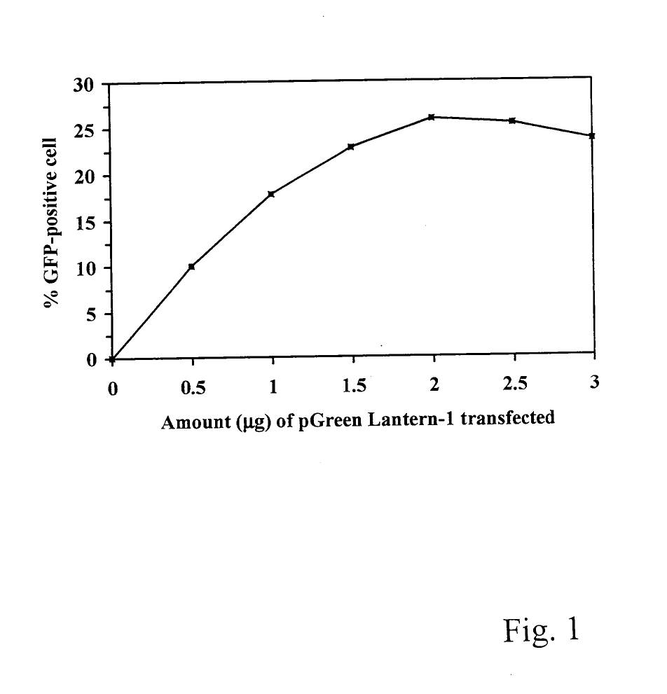

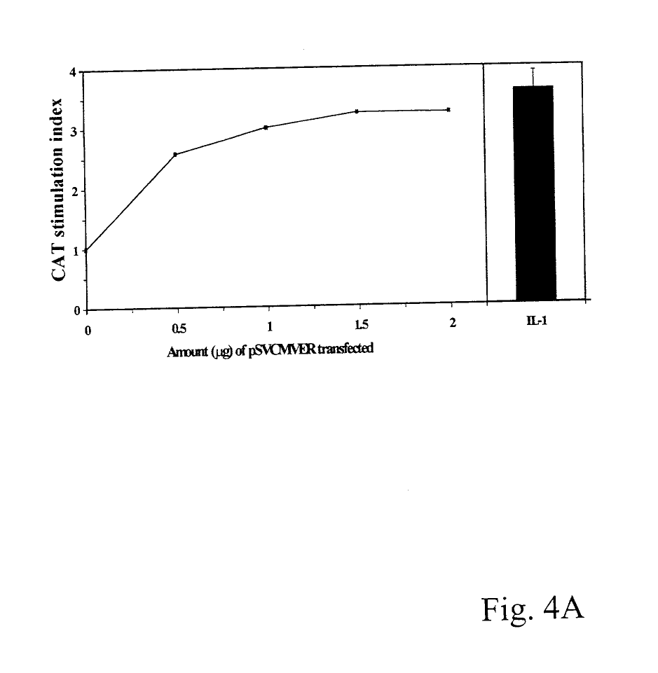

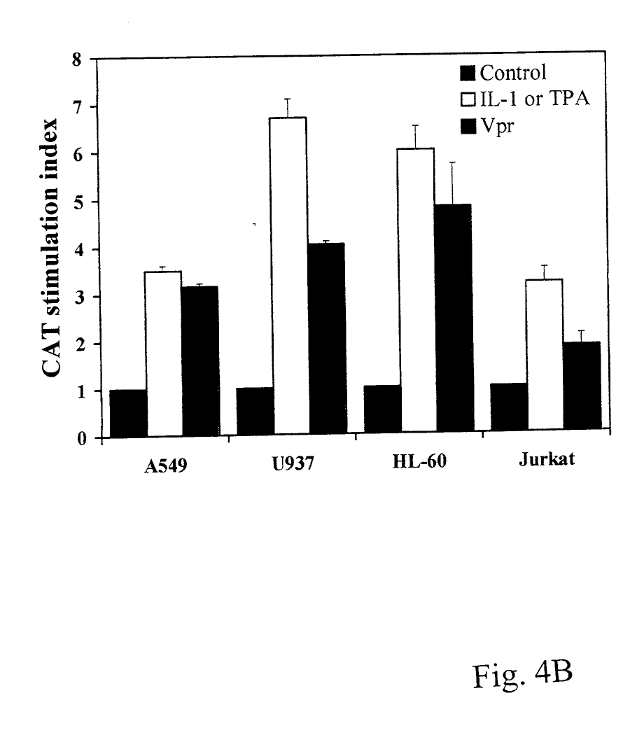

Transfections of A549 and COS-7 cells were performed using DMRIE-C (Gibco BRL) as recommended by the manufacturer. In brief, cells were seeded at 3.0×10⁵ cells per 35 mm well and were transfected 18-20 hours later with a total of 4 μg of plasmid DNA using 3 μl of DMRIE-C in a 1 ml volume of Opti-MEM I (Gibco BRL). Transfections were stopped after 6 hours by adding an equal volume of DMEM containing 20% heat-inactivated FCS. Twenty four hours after transfection, the media was replaced and the cells were incubated for an additional 24 hours. For the CAT ELISA, the media was replaced with serum-free DMEM with or without IL-1a. As demonstrated in FIG. 4A, the quantity (2 mg) of Vpr expressing vector (PSVCMVER) used for the cotransfection was shown to be optimal. Comparable optimization conditions with pgreen Lantern-1 (Gibco BRL) also used 2 mg of plasmid (FIG. 1). For the transfection of U937, HL-60 and Jurkat cells, the method was similar as for A549 cells, except that 1×10⁶ cells were used with 6 ml of DMRIE-C for a total of 6 mg of plasmid DNA.

In several experiments, Vpr expressing cells were enriched by affinity-purification following cotransfection with Vpr-expression plasmid and the pHook-3 system (Invitrogen, Carlsbad, Calif.). Cotransfection of pSVCMVER with the pHook-3 vector, which expresses and displays a single-chain antibody (sFv), allowed rapid isolation of transfected cells using hapten-coated magnetic beads. Briefly, 24 hours after the start of the cotransfection, the cells were harvested using 3 mM EDTA/phosphate-buffered saline (PBS) and incubated with the hapten-coupled magnetic beads for 60 minutes on a slow rotator at 37° C. in complete DMEM. The magnetic bead-bound cells were purified with a magnetic particle concentrator (Dynal, Lake Success, N.Y.), counted and seeded at 1×10⁴ cells per well (in 96-well dishes) in 200 ml of serum-free DMEM. The cells were incubated for an additional 18 hours before assaying the media for IL-8 protein.

IL-8 Assay.

Extracellular IL-8 protein concentrations were determined by ELISA (R&D Systems, Minneapolis, Minn.).

Chloramphenicol Acetyltransferase (CAT) Assay.

Levels of CAT enzyme were determined from 50 μg of cytoplasmic protein lysate using a CAT-specific ELISA (Boehringer Mannheim).

Electrophoretic Mobility Shift Assay (EMSA).

EMSA was performed using nuclear protein extracts from Vpr-transfected or Vpr-untransfected A549 cells, and procedures outlined by Scheinman, et al. (1995). In brief, nuclei were isolated after 24 hours from cells which were or were not transfected with pSVCMVER. Following gentle lysis of cell pellets in buffer containing 0.1% Nonidet P-40 and protease inhibitors PMSF, leupeptin and pepstatin A, nuclear proteins were isolated from nuclei by high salt buffer extraction (0.4M). Nuclear protein concentrations were determined calorimetrically with Bradford dye (BioRad, Mississauga, ON), and either stored at −80 until use, or used directly in our EMSA. Nuclear proteins were added at 5-10 μg/50 μl with ³²P-labeled DNA oligonucleotide probes to our EMSA buffer mix, followed by a 30 minute incubation on ice. Oligonucleotide probes used in our EMSA were based on both the published DNA sequence data for the IL-8 NF-κB and NF-IL-6 enhancer sequences and our positive CAT results. They were NF-IL-6 sequence: 5′ GGGAAGCTTCAGTTGCAAATCCTGGCAGTTGCAAATCGTGGGATCCGG (SEQ ID NO:13); NF-κB sequence: 5′ CCCGGATCCAGGAAATTCCCAGGAAATTCCAAGCTTCCC (SEQ ID NO:14); and NF-IL-6/NF-κB: 5′ CCCGGATCCAGGAAATTCCACGATTTGCAACTGAAGCTTCCC (SEQ ID NO:15).

Following the 30 minute incubation on ice, the ³²P-oligonucleotide/protein constructs were resolved in a 5% Tris-borate buffered polyacrylamide gel.

Imunofluorescence Analysis.

Transiently transfected A549 and COS-7 cells were washed twice in PBS and fixed on microscopic slides using 1:1 (v:v) acetone:methanol for 5 minutes at −20° C. Vpr expression was detected using a rabbit anti-Vpr serum (1:75) followed by a FITC-conjugated goat anti-rabbit serum (1:300, Cappel, Scarborough, ON). Cells were again washed and photographed using a Zeiss microscope equipped with epifluorescence.

Immunoblotting Analysis.

Protein samples (100 μg) derived from total lysate of the transfected cells were prepared in SDS sample buffer 48 hours posttransfection and resolved in a 15% SDS-polyacrylamide gel. The proteins were transferred onto a nitrocellulose membrane (Gelman Sciences, Ann Arbor, Mich.) and probed with a rabbit anti-Vpr serum (1:500). Bound antibody was detected with a horseradish peroxidase-labeled donkey anti-rabbit serum (1:3000, Amersham, Oakville, ON) developed using ECL Western blotting detection kit (Amersham).

Biological Results

Expression of Vpr

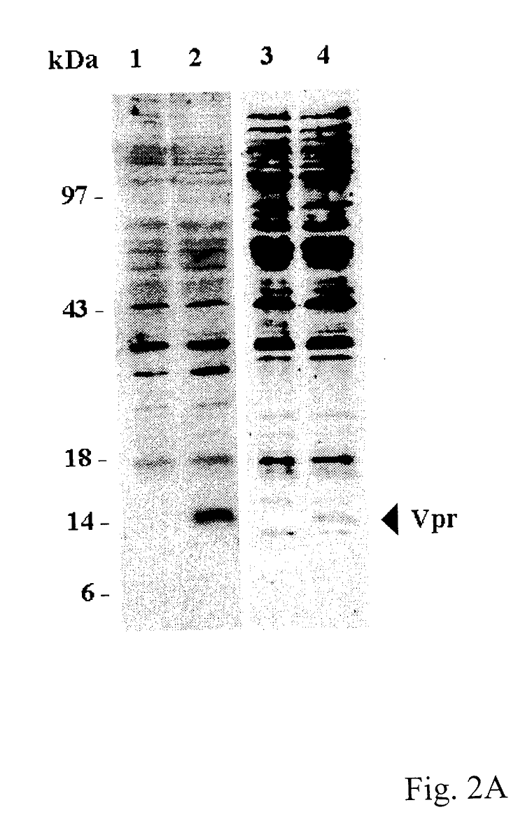

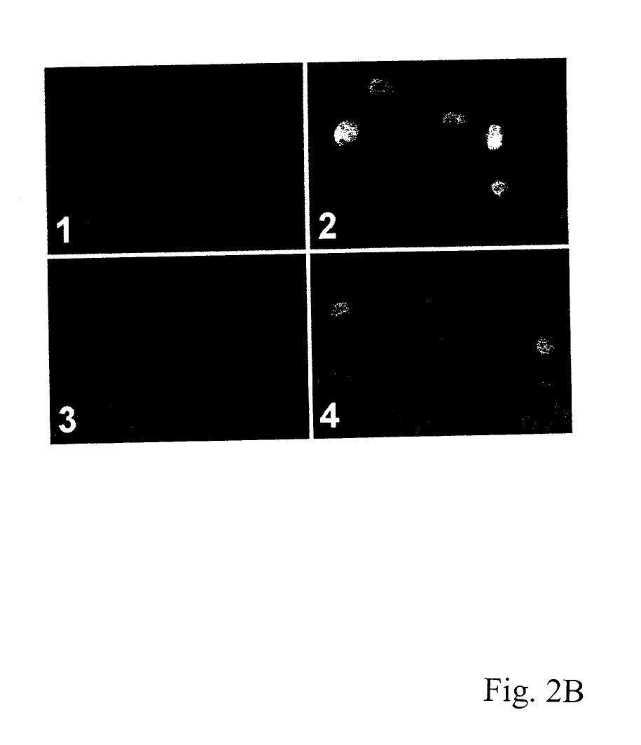

In order to study the biological properties of Vpr, the Vpr encoding DNA fragment from HIV strain ELI was cloned into the CMV IE promoter-based eucaryotic expression plasmid (see details in Materials and Methods). As shown in FIG. 2, transfection of COS-7 or A549 cells with Vpr expression plasmid followed by western blotting with anti-Vpr rabbit sera resulted in the detection of a unique 14 kDa immunoreactive band (FIG. 2A). The blot representing the A549 cells was overexposed in order to clearly identify the 14 kDa band corresponding to Vpr. In order to confirm that our pCMVdriven Vpr was properly targeted to the cell nucleus, immunofluorescence on acetone/methanol fixed Vpr-transfected cells was performed. Analysis of Vpr expression by fluorescence microscopy indicates that cells transfected with Vpr displayed specific nuclear fluorescence, whereas those cells that were transfected with vector displayed no Vpr-specific cellular immunofluorescence (FIG. 2B). These western blot and fluorescence microscopy results are in agreement with those previously reported for Vpr size and cellular localization.

Induction of IL-8 by Vpr in a IL-8-Inducible Cell Line.

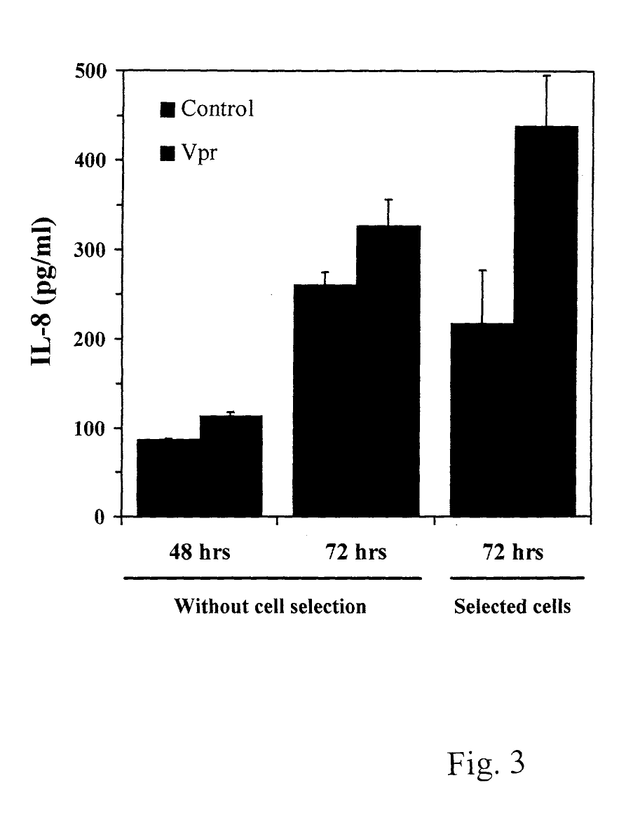

In order to determine whether Vpr was capable of inducing IL-8 in cells, Vpr expression plasmid was transfected into the IL-8 inducible cell line A549. The A549 cell line was chosen since this cell line has been shown to express IL-8 following IL-1 treatment, and has been successfully used to study the IL-8 promoter and protein. As shown in FIG. 3, Vpr increased the levels of extracellular protein by 30% and 26%, 48 and 72 hours posttransfection, respectively. Following enrichment of Vpr-expressing cells using the pHook-3 system, a 105% increase in IL-8 secretion was demonstrated (FIG. 3).

Identification of Vpr-Responsive Elements Required for Transcriptional Activation of the IL-8 Gene.

To characterize the DNA sequences involved in IL-8 gene activation by HIV Vpr, we constructed plasmids containing the full length IL-8 promoter or IL-8 promoter sequences deleted for parts of the IL-8 promoter 5′-flanking region fused to the bacterial CAT gene (FIG. 5A). We also used the cell line A549, since this cell line, like the monocytic cell lines used in this study, was not only capable of expressing IL-8 protein following IL-1 treatment, but responded with similar IL-8 enhancer elements as seen in monocytes following treatment with IL-1. As shown in FIG. 4A, transfection of A549 cells with increasing amounts of Vpr expression plasmid and full length IL-8 promoter/CAT construct resulted in a Vpr dose-dependent increase in IL-8 promoter driven CAT transcription. These results are comparable to those obtained for exogenous IL-1α (FIG. 4A).

Fine mapping of the IL-8 promoter/CAT construct indicated that the IL-8 promoter was responsive to Vpr, comparable to that seen for exogenous IL-1α (FIG. 4B) The -273/CAT construct was also capable of supporting Vpr induction of CAT activity, indicating that the sequences further upstream to this element (i.e. -1481 to -273) were dispensable for maximal IL-8 promoter activity. On the other hand, deletion of the NF-IL-6/NF-κB enhancer sequences (-94 to -72) abolished Vpr-stimulation of these CAT constructs. The slight increase in CAT activity observed when the IL-8- 1481 to -273 DNA sequences were linked to CAT might be due to the presence of the glucocorticoid responsive element, since this reporter plasmid was also found to be slightly responsive to dexamethasone (Roux, unpublished). To determine whether NF-IL-6/NF-κB together or alone were responsive to Vpr, CAT-reporter constructs containing each of these elements were cotransfected with Vpr expression plasmid into A549 cells (FIG. 5A). As seen in FIG. 5B, results indicate that either together or individually, NF-κB or NF-IL-6 enhancer sequences were able to respond to Vpr. Interestingly, IL-1α which is capable of inducing IL-8, acted only through the NF-κB enhancer, whereas Vpr acted on both enhancer sequences. When analyzed by EMSA both NF-κB and NF-IL-6 DNA binding sequences demonstrated significant gel shifts with protein extracts from cells transfected with Vpr, but no significant gel shift using protein extract from cells not transfected with Vpr (FIG. 5C). These results are in agreement with those previously published by Stein and Baldwin (1993) and Mukaida et al. (1994) who found that NF-IL-6 was respectively necessary or dispensable for optimal IL-8 promoter activity.

Analysis of Vpr stimulation of IL-8 promoter driven CAT activity in the monocytic cell lines U937 and HL-60, and in the T-lymphoid cell line Jurkat revealed a pattern of IL-1 and Vpr responsiveness both for the full length IL-8 promoter and the NF-IL-6/NF-κB enhancer sequences similar to that seen in A549 (FIG. 4B), suggesting that Vpr also induced IL-8 in monocytes through a comparable set of enhancer elements.

Enhancement of Cellular Promoters Containing NF-κB Enhancer Elements by Vpr

NF-IL-6 and NF-κB enhancer elements are found in numerous cellular promoter sequences, some of which are also proinflammatory cytokines. In order to determine whether Vpr was capable of acting on additional promoter sequences which contained NF-κB or NF-IL-6 elements, CAT-reporter plasmids containing these enhancer elements were assayed for transcriptional activation by Vpr. Results in table 1 indicate that Vpr was capable of enhancing several cellular and viral promoters including the HIV-LTR. Their induction appeared specific since the plasmid containing the thymidine kinase (TK) promoter revealed no marked increase in activity following co-transfection with Vpr.

TABLE 1
Stimulation of various viral and cellular enhancer-
promoter directed gene expression by the Vpr protein

| Enhancer-promoter | Control | Vpr |
|---|---|---|
| IL-8 | 1 | 3.33^{a} ± 0.25^{b} |
| IL-2 | 1 | 0.92 ± 0.05 |
| IL-2Ra | 1 | 1.75 ± 0.1 |
| HIV | 1 | 2.83 ± 0.05 |
| CMV | 1 | 4.18 ± 0.5 |
| SV40 | 1 | 2.26 ± 0.25 |
| TK | 1 | 1.18 ± 0.25 |

^{a}CAT activity is normalized to the activity obtained with control DNA-transfection.
^{b}Results represent the average of at least three independent transfections ± standard deviation.

### EXAMPLE 2

### Gene Therapy Using the DNA or RNA Constructs of the Present Invention

Plasmid DNA containing the IL-8 core VPR core responsive promoter elements comprised of the nucleotide sequence: AGTTGCAAATCGTGGAATTTCCTG (SEQ ID NO:16) and encoding NF-IL-6 and NFκB transcription protein recognition sequences are placed upstream of the enhancerless promoter of HSV TK (Luckow et al. (1987) Nucleic Acids Res. 15:5490). Immediately downstream of this promoter, the IL-2 cDNA or IL-6 nucleotide encoding sequences are placed. Primary antigen presenting cells obtained from the individual to be immunized are transfected via standard calcium phosphate or lipofectamine based transfection procedures and then reintroduced into the individual. Alternatively, plasmid are introduced into the individual by intramuscular injection or intranasally as an atomized spray in an inert carrier as naked DNA. This will induce a cell immunity based T cell helper-1 (THl) or T cell helper-2 (TH2) antibody based immunity with IL-6. Administration intranasally result in enhanced mucosal IgA immunity.

Viral based constructs comprised of either adenovirus or retrovirus vectors containing the VPR core responsive promoter elements comprised of the nucleotide sequence: AGTTGCAAATCGTGGAATTTCCTG (SEQ ID NO:16) and encoding NF-IL-6 and NFκB transcription protein recognition sequences are placed upstream of the enhancerless promoter of HSV TK (Luckow et al. (1987) Nucleic Acids Res. 15:5490) immediately down stream of this promoter, the IL-2 cDNA or alternative cytokine nucleotide encoding sequences are placed. Primary antigen presenting cells comprised of macrophage and dendritic cells are obtained from the individual to be immunized. This will induce a cell immunity based T cell helper-1 (THl) or T cell helper-2 (TH2) antibody based immunity with IL-6. Administration intranasally result in enhanced mucosal IgA immunity.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A DNA or RNA construct capable of expression of IL-2 in a warm-blooded animal or biological preparation, said recombinant DNA or RNA construct comprising:
a) a Vpr activated promoter;
b) a transcribable DNA segment coding for IL-2 and;
c) a secretory DNA encoding for a signal peptide functional in mammary cells and operably linked between said promoter and said DNA segment to facilitate secretion of said IL-2.

2. The DNA or RNA construct of claim 1 wherein said construct is recombinant.

3. The DNA or RNA construct of claim 2 wherein the promoter is NFκB, NF-IL-6, NFκB/NF-IL-6 or comprises the bases corresponding to -94 to -72 of the NFκB/NF-IL-6 enhancer sequence.

4. The DNA or RNA construct of claim 2 wherein the promoter is selected from the group consisting of:
a) NFκB recognition sequence:
AGGAAATTCCA (SEQ ID NO:1);
b) NF-IL-6 recognition sequence:
CAGTTGCAAATCGTG (SEQ ID NO:2);
c) NF-IL-6/NFκB recognition sequence:
AGTTGCAAATCGTGGAATTTCCTGAA (SEQ ID NO:3); and
d) -94 to -72 of the IL-8 core enhancer element NF-IL-6/NFκB recognition sequence:
CAGTTGCAAATCGTGGAATTTCC (SEQ ID NO:4).

5. The DNA or RNA construct as defined in claim 4 wherein said transcribable IL-2 has the following sequence:

| | | |
|---|---|---|
| CCCCATAATA TTTTTCCAGA ATTAACAGTA TAAATTGCAT CTCTTGTTCA AGAGTTCCCT | 60 | (SEQ ID NO:5) |
| ATCACTCTCT TTAATCACTA CTCACAGTAA CCTCAACTCC TGCCACAATG TACAGGATGC | 120 | |
| AACTCCTGTC TTGCATTGCA CTAAGTCTTG CACTTGTCAC AAACAGTGCA CCTACTTCAA | 180 | |
| GTTCTACAAA GAAAACACAG CTACAACTGG AGCATTTACT GCTGGATTTA CAGATGATTT | 240 | |
| TGAATGGAAT TAATAATTAC AAGAATCCCA AACTCACCAG CATGCTCACA TTTAAGTTTT | 300 | |
| ACATGCCCAA GAAGGCCACA GAACTGAAAC ATCTTCAGTG TCTAGAAGAA GAACTCAAAC | 360 | |
| CTCTGGAGGA AGTGCTAAAT TTAGCTCAAA GCAAAAACTT TCACTTAAGA CCCAGGGACT | 420 | |
| TAATCAGCAA TATCAACGTA ATAGTTCTGG AACTAAAGGG ATCTGAAACA ACATTCATGT | 480 | |
| GTGAATATGC TGATGAGACA GCAACCATTG TAGAATTTCT GAACAGATGG ATTACCTTTT | 540 | |
| GTCAAAGCAT CATCTCAACA CTGACTTGAT AATTAAGTGC TTCCCACTTA AAACATATCA | 600 | |
| GGCCTTCTAT TTATTTAAAT ATTTAAATTT TATATTTATT GTTGAATGTA TGGTTTGCTA | 660 | |
| CCTATTGTAA CTATTATTCT TAATCTTAAA ACTATAAATA TGGATCTTTT ATGATTCTTT | 720 | |
| TTGTAAGCCC TAGGGGCTCT AAAATGGTTT CACTTATTTA TCCCAAAATA TTTATTATTA | 780 | |
| TGTTGAATGT TAAATATAGT ATCTATGTAG ATTGGTTAGT AAAACTATTT AATAAATTTG | 840 | |
| ATAA | 844 | |

6. The DNA or RNA construct as defined in claim 5 wherein said transcribable further comprises a trancribable segment of IL-8.

7. The DNA or RNA construct as defined in claim 6 wherein the transcribable IL-8 has the following sequence:

| | | |
|---|---|---|
| GAATTCAGTA ACCCAGGCAT TATTTTATCC TCAAGTCTTA GGTTGGTTGG AGAAAGATAA | 60 | (SEQ ID NO:6) |
| CAAAAAGAAA CATGATTGTG CAGAAACAGA CAAACCTTTT TGGAAAGCAT TTGAAAATGG | 120 | |
| CATTCCCCCT CCACAGTGTG TTCACAGTGT GGGCAAATTC ACTGCTCTGT CGTACTTTCT | 180 | |
| GAAAATGAAG AACTGTTACA CCAAGGTGAA TTATTTATAA ATTATGTACT TGCCCAGAAG | 240 | |
| CGAACAGACT TTTACTATCA TAAGAACCCT TCCTTGGTGT GCTCTTTATC TACAGAATCC | 300 | |
| AAGACCTTTC AAGAAAGGTC TTGGATTCTT TTCTTCAGGA CACTAGGACA TAAAGCCACC | 360 | |
| TTTTTATGAT TTGTTGAAAT TTCTCACTCC ATCCCTTTTG CTGATGATCA TGGGTCCTCA | 420 | |
| GAGGTCAGAC TTGGTGTCCT TGGATAAAGA GCATGAAGCA ACAGTGGCTG AACCAGAGTT | 480 | |
| GGAACCCAGA TGCTCTTTCC ACTAAGCATA CAACTTTCCA TTAGATAACA CCTCCCTCCC | 540 | |
| ACCCCAACCA AGCAGCTCCA GTGCACCACT TTCTGGAGCA TAAACATACC TTAACTTTAC | 600 | |
| AACTTGAGTG GCCTTGAATA CTGTTCCTAT CTGGAATGTG CTGTTCTCTT TCATCTTCCT | 660 | |
| CTATTGAAGC CCTCCTATTC CTCAATGCCT TGCTCCAACT GCCTTTGGAA GATTCTGCTC | 720 | |
| TTATGCCTCC ACTGGAATTA ATGTCTTAGT ACCACTTGTC TATTCTGCTA TATAGTCAGT | 780 | |
| CCTTACATTG CTTTCTTCTT CTGATAGACC AAACTCTTTA AGGACAAGTA CCTAGTCTTA | 840 | |
| TCTATTTCTA GATCCCCCAC ATTACTCAGA AAGTTACTCC ATAAATGTTT GTGGAACTGA | 900 | |
| TTTCTATGTG AAGACATGTG CCCCTTCACT CTGTTAACTA GCATTAGAAA AACAAATCTT | 960 | |
| TTGAAAAGTT GTAGTATGCC CCTAAGAGCA GTAACAGTTC CTAGAAACTC TCTAAAATGC | 1020 | |
| TTAGAAAAAG ATTTATTTTA AATTACCTCC CCAATAAAAT GATTGGCTGG CTTATCTTCA | 1080 | |
| CCATCATGAT AGCATCTGTA ATTAACTGAA AAAAAATAAT TATGCCATTA AAAGAAAATC | 1140 | |
| ATCCATGATC TTGTTCTAAC ACCTGCCACT CTAGTACTAT ATCTGTCACA TGGTCTATGA | 1200 | |
| TAAAGTTATC TAGAAATAAA AAAGCATACA ATTGATAATT CACCAAATTG TGGAGCTTCA | 1260 | |
| GTATTTTAAA TGTATATTAA AATTAAATTA TTTTAAAGAT CAAAGAAAAC TTTCGTCATA | 1320 | |
| CTCCGTATTT GATAAGGAAC AAATAGGAAG TGTGATGACT CAGGTTTGCC CTGAGGGGAT | 1380 | |
| GGGCCATCAG TTGCAAATCG TGGAATTTCC TCTGACATAA TGAAAAGATG AGGGTGCATA | 1440 | |
| AGTTCTCTAG TAGGGTGATG ATATAAAAAG CCACCGGAGC ACTCCATAAG GCACAAACTT | 1500 | |
| TCAGAGACAG CAGAGCACAC AAGCTTCTAG GACAAGAGCC AGGAAGAAAC CACCGGAAGG | 1560 | |
| AACCATTCTC ACTGTGTGTA AACATG | 1586 | |

8. The DNA or RNA construct as defined in claim 7 wherein the signal peptide is an IL-2 signal peptide or an analogue or derivative thereof.

9. The DNA or RNA construct as defined in claim 8 wherein the IL-2 signal peptide has the following sequence:

| Met Tyr Arg Met Gln Leu Leu Ser Cys Ile Ala Leu Ser Leu Ala Leu | (SEQ ID NO:7) |
|---|---|
| 1 5 10 15 | |
| Val Thr Asn Ser Ala Pro Thr Ser Ser Ser | |
| 20 25 | |

10. A method for increasing the immune response of a warm-blooded animal or biological preparation comprising the steps of:
a) introducing a DNA or RNA construct as defined in claim 1 in stem cells, antigen presenting cells or immune cell leukocytes, fibroblasts and epithelial cells, of the warm-blooded animal or biological preparation to obtain a transfected cell populations; and
b) administering a pharmaceutically effective amount of said transfected cell populations to the warm-blooded animal or biological preparation.

11. The method of claim 10 wherein said warm-blooded animal is an immunocompromised patient.

12. A method for inhibiting expression of IL-8 of a warm-blooded animal or a biological preparation comprising the step of administering a pharmaceutically effective amount of a Vpr inhibitor.

13. The method of claim 12 wherein said warm-blooded animal is an immunocompromised patient.

14. The method of claim 13 wherein the Vpr inhibitor is an anti-Vpr antibody.

15. The method of claim 14 wherein the anti-Vpr antibody is a monoclonal antibody.

16. A method for stimulating IL-8 expression in a mammal in need to, said method comprising the step of administering a pharmaceutically effective amount of a pharmaceutically acceptable formulation comprising a Vpr protein to said mammal.

17. A method for determining the interaction between Vpr and other proteins, said method comprises the steps of:
a) co-precipitation of Vpr and associated cellular proteins using anti-Vpr antibody followed by protein gel electrophoresis;
b) development of a yeast two hybrid system in which a Vpr-Gal4 construct is introduced into yeast to screen human cDNAs expressed in a yeast library and detection of Gal4 insensitive colonies; and
c) constriction of Vpr deletion mutants to identify both association of cellular proteins with Vpr or Vpr subdomains.


SEQUENCE LISTING (52 sequences)

SEQ ID NO: 1 - DNA, 21 bp, Artificial sequence
  primer
caagtctgtgacttgcacgta

SEQ ID NO: 2 - DNA, 21 bp, Artificial sequence
  primer
ttcttgcggagattctcttcc

SEQ ID NO: 3 - DNA, 30 bp, Artificial sequence
  primer
ccggaattcatgtcaaacgtgcgagtgtct

SEQ ID NO: 4 - DNA, 32 bp, Artificial sequence
  primer
ccggaattcttacgtttgacgtcttctgaggc

SEQ ID NO: 5 - DNA, 20 bp, Artificial sequence
  primer
gctgtgctatccctgtacgc

SEQ ID NO: 6 - DNA, 19 bp, Artificial sequence
  primer
ccaatggtgatgacctggc

SEQ ID NO: 7 - DNA, 21 bp, Artificial sequence
  primer
ctacaagcagtcacagcacat

SEQ ID NO: 8 - DNA, 21 bp, Artificial sequence
  primer
ctaactctgaggacacgcatt

SEQ ID NO: 9 - DNA, 20 bp, Artificial sequence
  primer
cgagaagctgtgctacgtcg

SEQ ID NO: 10 - DNA, 30 bp, Artificial sequence
  primer
gcgcggatccacagcgatatccagacattc

SEQ ID NO: 11 - DNA, 26 bp, Artificial sequence
  primer
gcggggatcccgtgggcacttcaggg

SEQ ID NO: 12 - DNA, 27 bp, Artificial sequence
  primer
gggaggtacccgcacttgcaatatgac

SEQ ID NO: 13 - DNA, 30 bp, Artificial sequence
  primer
atttggtaccgttatttccattcggatgcc

SEQ ID NO: 14 - DNA, 22 bp, Artificial sequence
  primer
tgtgatggtgggaatgggtcag

SEQ ID NO: 15 - DNA, 26 bp, Artificial sequence
  primer
tttgatgtcacgcacgcacgatttcc

SEQ ID NO: 16 - DNA, 20 bp, Artificial sequence
  primer
ccacagctgggagatttagc

SEQ ID NO: 17 - DNA, 20 bp, Artificial sequence
  primer
acccttgtcaccatctcagg

SEQ ID NO: 18 - DNA, 20 bp, Artificial sequence
  primer
ggtcacttcatgcctgtcct

SEQ ID NO: 19 - DNA, 20 bp, Artificial sequence
  primer
aacctgtccctccagaacct

SEQ ID NO: 20 - DNA, 20 bp, Artificial sequence
  primer
tggacctggaggaaatcttg

SEQ ID NO: 21 - DNA, 20 bp, Artificial sequence
  primer
cgcctacgactcagatgtca

SEQ ID NO: 22 - DNA, 20 bp, Artificial sequence
  primer
ggggtccttatccatccact

SEQ ID NO: 23 - DNA, 20 bp, Artificial sequence
  primer
caaccacatctcctccctgt

SEQ ID NO: 24 - DNA, 20 bp, Artificial sequence
  primer
caactggacagcacctcttg

SEQ ID NO: 25 - DNA, 20 bp, Artificial sequence
  primer
ttgcccagtatggagatgtg

SEQ ID NO: 26 - DNA, 20 bp, Artificial sequence
  primer
cacgaagccagtgcagataa

SEQ ID NO: 27 - DNA, 19 bp, Artificial sequence
  primer
ggagaactccgggacctgt

SEQ ID NO: 28 - DNA, 20 bp, Artificial sequence
  primer
aacctgtgcctttgtggaag

SEQ ID NO: 29 - DNA, 20 bp, Artificial sequence
  primer
atgactgagaggctccgaga

SEQ ID NO: 30 - DNA, 19 bp, Artificial sequence
  primer
aaagagaggccatcatcct

SEQ ID NO: 31 - DNA, 20 bp, Artificial sequence
  primer
tctctgtggcgtgttctctg

SEQ ID NO: 32 - DNA, 20 bp, Artificial sequence
  primer
tgattttgcaaatgctccag

SEQ ID NO: 33 - DNA, 20 bp, Artificial sequence
  primer
gcttcaggatgaaggacagg

SEQ ID NO: 34 - DNA, 20 bp, Artificial sequence
  primer
ggtgaaggtcggtgtcaacg

SEQ ID NO: 35 - DNA, 20 bp, Artificial sequence
  primer
ctgggagcaagtccagagag

SEQ ID NO: 36 - DNA, 20 bp, Artificial sequence
  primer
ttgccatctcttgtttgctg

SEQ ID NO: 37 - DNA, 20 bp, Artificial sequence
  primer
ggatgacattccacctggtt

SEQ ID NO: 38 - DNA, 20 bp, Artificial sequence
  primer
tgggtcaaactccaactgtg

SEQ ID NO: 39 - DNA, 20 bp, Artificial sequence
  primer
ggggtatccgtgtagcacat

SEQ ID NO: 40 - DNA, 20 bp, Artificial sequence
  primer
ggaaggccaaagagaagagg

SEQ ID NO: 41 - DNA, 20 bp, Artificial sequence
  primer
gggatgttaccccatgacac

SEQ ID NO: 42 - DNA, 20 bp, Artificial sequence
  primer
tcccacctctcattctccac

SEQ ID NO: 43 - DNA, 20 bp, Artificial sequence
  primer
caaattcagggccagacagt

SEQ ID NO: 44 - DNA, 20 bp, Artificial sequence
  primer
gaacactgctcgtggtttca

SEQ ID NO: 45 - DNA, 20 bp, Artificial sequence
  primer
ggaaattccgctgtgctaag

SEQ ID NO: 46 - DNA, 20 bp, Artificial sequence
  primer
ggaaccacaaggacctcaaa

SEQ ID NO: 47 - DNA, 20 bp, Artificial sequence
  primer
agctgatcgaagtgggtgtc

SEQ ID NO: 48 - DNA, 40 bp, Artificial sequence
  primer
acctaaggagtggccgaactcaggaacctggaaggacaga

SEQ ID NO: 49 - DNA, 20 bp, Artificial sequence
  primer
cactgcagcccctcactatt

SEQ ID NO: 50 - DNA, 20 bp, Artificial sequence
  oligonucleotide for hybridization
tggttctcaacgtgtccttg

SEQ ID NO: 51 - DNA, 20 bp, Artificial sequence
  oligonucleotide for hybridization
gaaattcctgaaaccgacca

SEQ ID NO: 52 - DNA, 20 bp, Artificial sequence
  oligonucleotide for hybridization
caaagttgtcatggatgacc